(19) United States
(12) Patent Application Publication
(10) Pub. No.: US 2021/0346248 A1
(43) Pub. Date: Nov. 11, 2021

(54) COSMETIC COMPOSITION IN THE FORM OF A REVITALIZING AND TONING OIL-IN-WATER EMULSION FOR SKIN AND LIPS

(71) Applicant: CONTRAD SWISS SA, Lugano (CH)

(72) Inventor: Joanne SERRENTINO, Sainte-Catherine-de-Hatley (CA)

(21) Appl. No.: 17/315,630

(22) Filed: May 10, 2021

(30) Foreign Application Priority Data
May 11, 2020 (CH) 00566/20

(51) Int. Cl. A61K 8/06; A61K 8/67; A61K 8/65; A61K 8/34; A61K 8/44; A61Q 19/00

(52) U.S. Cl. CPC A61K 8/062; A61K 8/673; A61K 8/65; A61K 8/345; A61K 8/44; A61K 2800/592; A61K 2800/48; A61K 2800/51; A61K 2800/522; A61K 2800/524; A61Q 19/001

(57) ABSTRACT

A cosmetic composition in the form of an oil-in-water emulsion for topical application to skin and/or lips, having a revitalizing and toning effect on skin and lips is provided. The cosmetic composition comprises a biomimetic peptide, hydrolysed collagen, panthenol, and one or more additional optional ingredients selected from the group consisting of rheological agents, emulsifying agents, wetting agents, emollient agents, skin conditioning agents, antioxidant agents, chelating agents, preservative agents, pH adjusting agents, and any combination thereof.

### CROSS REFERENCE TO RELATED APPLICATION

This application claims priority to and benefit of Swiss Patent Application No. 00566/20, filed on May 11, 2020, which is fully incorporated by reference herein in its entirety.

### FIELD OF THE INVENTION

The present invention generally relates to the cosmetic field and more precisely to a cosmetic composition in the form of an oil-in-water emulsion with a protective and restructuring action, having a high moisturizing and toning action on the skin and lips.

The composition of the present invention contains a specific biomimetic oligopeptide capable of exerting a moisturizing, toning and volumizing action on the skin and lips, particularly when combined with the additional ingredients present in the composition.

### BACKGROUND OF THE INVENTION

Oligopeptides generally have a molecular weight of less than 5,000 Daltons and consist of an amino acid chain which “mimics” active domains of proteins normally found in the human body. They are in fact called “biomimetic” oligopeptides since they can bind to specific cellular receptors, thereby modulating several biological processes. Each oligopeptide has a specific activity; particularly on the skin and lips, they can perform stimulating, muscle relaxant, remodelling, repairing, strengthening, lightening, desensitizing, and many other functions. Their use is usually recommended from 35-40 years of age, depending on the state of skin ageing. The use of oligopeptides, in addition to their proven functionality, is accompanied by the undoubted advantage of total safety of use as they faithfully mimic parts of molecules normally found in the human body. The possibilities for the development of biomimetic peptides are countless, as well as the possibilities for the creation of a mix of peptides with specific and, in any case, complementary actions, or for the selection of specific oligopeptides according to the specific activity required.

### SUMMARY OF THE INVENTION

The present invention provides a cosmetic composition in the form of an oil-in-water emulsion for topical application to the skin and/or lips, which comprises a highly functional combination of ingredients for the well-being of the skin and lips.

Advantages of the cosmetic composition according to the present invention are also described.

As illustrated in detail hereinafter, thanks to its particular combination of ingredients, as well as its pH and absorption characteristics, the cosmetic composition in the form of an oil-in-water emulsion of the present invention is particularly effective in enhancing hydration and elasticity of skin and lips, achieving an immediate and sustained volumizing effect on the lips.

### DETAILED DESCRIPTION

The cosmetic composition in the form of an oil-in-water emulsion according to the present invention comprises the combination of the following components:

a peptide component, consisting of a biomimetic oligopeptide designated as SH-Polypeptide 47,

hydrolysed collagen, and

panthenol.

SH-Polypeptide 47 is a single-chain recombinant human peptide having a maximum length of 1464 amino acids and comprising the amino acid sequence SEQ ID NO:1. It may include disulfide bridges and/or glycosylation. SH-Polypeptide 47 is manufactured through a recombinant technique known per se, including, for example, fermentation by a prokaryotic expression system, such as E. coli, by using a starting gene which is the synthetic copy of the human gene encoding for Collagen alpha 1, used as such or adapted to the expression system used as the host.

According to a preferred embodiment, SH-Polypeptide 47 consists of the amino acid sequence of SEQ ID No: 1.

SEQ ID NO: 1:
SAGFDFSFLP QPPQEKAHDG GRYYRA

Collagen is the most abundant protein family in humans, representing the main component of the connective tissue. Among the various types of collagen, the main component of the dermis is type I collagen characterized by a heterotrimeric structure comprising two alpha 1 polypeptide chains and one alpha 2 polypeptide chain joined together to form a triple helix. It primarily exerts a structural support function and plays a central role in maintaining the skin's compactness, elasticity, and firmness. With ageing, collagen is synthesized less and degraded more, resulting in gradual loss of skin tone and plasticity and in the appearance of wrinkles.

In the present description, the term “hydrolysed collagen” refers to a mixture of peptide fragments, which are obtained by controlled chemical or enzymatic hydrolysis of the native collagen molecule and are characterized by a molecular weight between approximately 3,000 and 6,000 Daltons and a high content of proline and hydroxyproline.

In its hydrolysed form, collagen has several beneficial effects on tissues, particularly on the skin, by stimulating the natural regeneration of fibroblasts and their synthesis of new collagen, while inhibiting the proteolytic action exerted on this protein by tissue metalloproteinases. The activity of hydrolysed collagen, in synergy with the other ingredients, therefore, contributes to the restructuring and toning effect of the cosmetic composition of the present invention on the skin and lips.

The cosmetic composition according to the invention also contains panthenol (provitamin B5). This molecule is a precursor of vitamin B5 which contributes to a filming, soothing and protective action on the skin. Therefore, it may be considered as a valid support for the skin cell-regenerating activity carried out by the oligopeptide defined above.

According to the present invention, the cosmetic composition is in the form of an oil-in-water emulsion, thereby resulting particularly smooth-flowing and easy to apply.

In one embodiment, the cosmetic composition of the present invention is a cream.

In a preferred embodiment, the cosmetic composition of the present invention also comprises a rheological agent capable of increasing the stability of the emulsion and favouring its flow by modifying the viscosity thereof. Rheological agents particularly suitable for use in the cosmetic composition of the present invention are acrylic polymers and derivatives thereof, such as carboxyvinyl polymers known under the trade name of Carbomer, which identifies a family of high-molecular-weight cross-linked acrylic acid homopolymers.

In another preferred embodiment, the cosmetic composition of the present invention also comprises an emulsifying agent capable of promoting the stability thereof. Exemplary emulsifying agents suitable for use in the cosmetic composition of the present invention are cetyl stearyl alcohol, dimethicone, sodium stearoyl glutamate, C12-20 Acid PEG-8 Ester (CAS number: 68908-68-9), Ceteareth-20 and/or Ceteareth-12 (CAS number: 68439-49-6).

According to further preferred embodiments, the cosmetic composition of the present invention also contains one or more of the following additional components: wetting agents, emollient agents, skin conditioning agents, antioxidant agents, chelating agents, preservative agents, pH adjusting agents, and any combination thereof.

An exemplary wetting agent suitable for use in the cosmetic composition of the present invention is glycerin, a lipid component physiologically present in human skin with moisturizing and softening properties.

Exemplary emollient agents suitable for use in the cosmetic composition of the present invention are cyclopentasiloxane, cetyl stearyl isononanoate and/or sodium polyacrylate. These ingredients are characterized by a significant film-forming activity, thus ensuring sustained moisturizing and softening effects of the formulation on the skin and lips.

Skin conditioners in the cosmetic composition according to the present invention contribute to the well-being of the skin and lips by promoting, i.a., maintenance or restoration of the lipid barrier and reducing water loss. Examples of skin conditioners suitable for use in the cosmetic composition of the present invention are Aloe barbadensis leaf extract, vegetable oils, retinyl palmitate, and sodium hyaluronate. The latter is a hyaluronic acid salt which has very important hygroscopic characteristics and helps improve the skin barrier function and consequently hydration of the skin itself.

Preferably, the vegetable oil used in the cosmetic composition according to the present invention is selected from the group consisting of Prunus amygdalus dulcis (Sweet Almond) oil, Persea gratissima (Avocado) oil, Helianthus annuus (Sunflower) seed oil, Argania spinosa (Argan) kernel oil, and any mixture thereof. These substances have moisturizing, soothing and elasticizing actions on the skin and lips and are able to stimulate the synthesis of collagen and elastin.

Exemplary antioxidant agents suitable for use in the cosmetic composition of the present invention are tocopherol (vitamin E) and esters thereof, preferably tocopheryl acetate. These ingredients are particularly effective in counteracting the activity of free radicals, exerting a strong antioxidant action on the skin, and protecting cell membranes and the integrity of collagen fibres.

An exemplary chelating agent suitable for use in the cosmetic composition of the present invention is ethylenediaminetetraacetic acid (EDTA) disodium salt, which is suitable to assist the preservative system by acting as a heavy metal chelator.

Exemplary preservatives suitable for use in the cosmetic composition of the present invention are phenoxyethanol acid, dimethylol dimethyl hydantoin (DMDM hydantoin), sodium benzoate and/or potassium sorbate; particularly if used in a mixture, they can prevent any type of bacterial and/or fungal contamination of the formulation.

Exemplary pH adjusting agents suitable for use in the cosmetic composition of the present invention are sodium hydroxide and citric acid, which are introduced in quantities to adjust the pH of the formulation within the identified preferred range (pH between 5.00 and 6.00).

The preferred embodiments described above may be combined with each other, and the implementation of such combinations falls within the skills of the persons skilled in the art.

The table below illustrates, by way of non-limiting example, suitable percentages (% w/w) of the ingredients of a cosmetic composition falling within the scope of the present invention:

| Ingredient | % w/w |
|---|---|
| Water | >60 |
| Cetyl stearyl alcohol | 1-5 |
| Dimethicone | 1-5 |
| Cyclopentasiloxane | 1-5 |
| Hydrolysed collagen | 1-5 |
| C12-20 Acid PEG-8 Ester | 1-5 |
| Glycerin | 1-5 |
| Ceteareth-12 | 1-5 |
| Aloe barbadensis leaf extract | 1-5 |
| Ceteareth-20 | 1-5 |
| Prunus amygdalus dulcis (Sweet almond) oil | 1-5 |
| Cetyl stearyl isononanoate | 1-5 |
| Sodium stearoyl glutamate | 0.1-1 |
| Phenoxyethanol | 0.1-1 |
| Carbomer | 0.1-1 |
| Panthenol | 0.1-1 |
| Tocopheryl acetate | 0.1-1 |
| DMDM hydantoin | 0.1-1 |
| Sodium hyaluronate | 0.1-1 |
| Persea gratissima (Avocado) oil | 0.1-1 |
| EDTA disodium salt | 0.1-1 |
| Sodium hydroxide | 0.01-1 |
| Retinyl palmitate | 0.01-1 |
| Helianthus annuus (Sunflower) seed oil | 0.01-1 |
| Citric acid | 0.01-1 |
| Argania spinosa (Argan) kernel oil | 0.01-1 |
| Sodium chloride | 0.0000001-0.01 |
| Sodium benzoate | 0.0000001-0.01 |
| Potassium sorbate | 0.0000001-0.01 |
| SH-Polypeptide 47 | 0.0000001-0.01 |
| Tocopherol | 0.0000001-0.01 |
| Sodium polyacrylate | 0.0000001-0.01 |

### Experimental Section

A formulation comprising the ingredients listed in the above table and falling within the quantitative ranges indicated therein was subjected to a moisturizing, elasticizing and volumizing efficacy study on 20 volunteers aged between 18 and 60. The study was performed under the supervision of a specialist in Dermatology and Venereology and included the following assessments:

### 1) Elasticizing Activity

### Treatment Area: Lips

At T0 and T7 days from the application of the product twice a day, skin elasticity measurements are carried out through an Elastimeter (Delfin Technologies Ltd.) providing the values in Newtons/m.

### 2) Moisturizing Activity

### Treatment Area: Lips

Corneometric measurements are carried out at T0, T20, T60 and T240 minutes and T7 days (instrument used MoistureMeterSC, Delfin Technologies Ltd.) in order to assess the moisturizing activity of the product.

### 3) Volumizing Activity

### Treatment Area: Lips

Measurements of the area occupied by the lips (in mm²) are carried out using a high-resolution camera (Antera 3D) and the software CS (Miravex Ltd.) in order to assess the volumizing activity of the product.

The volumizing effectiveness is measured by comparing the area occupied by the lips at the end of the treatment period with the area measured before starting the treatment.

### Results

The results of clinical tests showed that the formulation object of the present invention caused:

an increase in the volume of the lips measured after 20 minutes (T20), 60 minutes (T60) and 7 days (T7) from the application of the product. In particular, the volume of the lips treated with the product is increased by a value equal to 1.3% after 20 minutes, a value equal to 1.2% after 60 minutes, and a value equal to 1.7% after 7 days from the application; these changes are statistically significant (p<0.05);

an increase in lip hydration after 20 minutes (T20) and 60 minutes (T60) from the application of the product. In particular, the hydration of the lips treated with the product is increased by a value equal to 24% after 20 minutes and a value equal to 13.3% after 60 minutes from the application; these changes are statistically significant (p<0.05); and

an increase in lip elasticity after 7 days (T7) from the application of the product. In particular, the elasticity of the lips treated with the product is increased by a value equal to 26% after 7 days from the application, and this change is statistically significant (p<0.05).

What is claimed is:

1. A cosmetic composition in the form of an oil-in-water emulsion for topical application to skin and/or lips, comprising:
a peptide component, consisting of a single-chain recombinant human peptide having a maximum length of 1464 amino acids and comprising the amino acid sequence SEQ ID NO:1,
hydrolysed collagen, and
panthenol.

2. The cosmetic composition of claim 1, wherein the cosmetic composition is a cream.

3. The cosmetic composition of claim 1, wherein the single-chain recombinant human peptide consists of the amino acid sequence SEQ ID NO:1.

4. The cosmetic composition of claim 1, further comprising a rheological agent.

5. The cosmetic composition of claim 1, further comprising an emulsifying agent.

6. The cosmetic composition of claim 5, wherein the emulsifying agent is selected from the group consisting of cetyl stearyl alcohol, dimethicone, sodium stearoyl glutamate, C12-20 Acid PEG-8 Ester, Ceteareth-12, Ceteareth-20, and any combination thereof.

7. The cosmetic composition of claim 1, further comprising additional ingredients selected from the group consisting of wetting agents, emollient agents, skin conditioning agents, antioxidant agents, chelating agents, preservative agents, pH adjusting agents, and any combination thereof.

8. The cosmetic composition of claim 7, comprising glycerin as wetting agent.

9. The cosmetic composition of claim 7, wherein the emollient agents are selected from the group consisting of cyclopentasiloxane, cetyl stearyl isononanoate, sodium polyacrylate, and any combination thereof.

10. The cosmetic composition of claim 7, wherein the skin conditioning agents are selected from the group consisting of Aloe barbadensis leaf extract, Prunus amygdalus dulcis (Sweet almond) oil, Persea gratissima (Avocado) oil, Helianthus annuus (Sunflower) seed oil, Argania spinosa (Argan) kernel oil, retinyl palmitate, sodium hyaluronate, and any combination thereof.

11. The cosmetic composition of claim 7, wherein the antioxidant agents are tocopherol and/or an ester thereof.

12. The cosmetic composition of claim 7, wherein the chelating agent is EDTA disodium salt.

13. The cosmetic composition of claim 7, wherein the preservative agents are selected from the group consisting of phenoxyethanol, dimethylol dimethyl hydantoin (DMDM hydantoin), sodium benzoate, potassium sorbate, and any combination thereof.

14. The cosmetic composition of claim 7, wherein the pH adjusting agents are sodium hydroxide and/or citric acid.

15. The cosmetic composition of claim 1, said cosmetic composition having a pH between 5.00 and 6.00.

16. A method for moisturizing, elasticizing and volumizing skin and/or lips, the method comprising topically applying to the skin and/or lips the cosmetic composition in the form of an oil-in-water emulsion of claim 1.


SEQUENCE LISTING (1 sequence)

SEQ ID NO: 1 - protein, 26 aa, Homo sapiens
SAGFDFSFLPQPPQEKAHDGGRYYRA